(19) United States
(12) Patent Application Publication
(10) Pub. No.: US 2014/0276094 A1
(43) Pub. Date: Sep. 18, 2014

(54) APPARATUS FOR OPTICAL SCANNING OF THE FOOT FOR ORTHOSIS

(71) Applicant: Roy Herman Lidtke, Elberon, IA (US)

(72) Inventor: Roy Herman Lidtke, Elberon, IA (US)

(21) Appl. No.: 13/836,724

(22) Filed: Mar. 15, 2013

(51) Int. Cl. A61B 5/107; A61B 5/00

(52) U.S. Cl. CPC A61B 5/1079; A61B 5/0064; A61B 5/1074; A61B 5/1078
USPC 600/476

(57) ABSTRACT

An apparatus is provided which provides proper compression of the soft tissue to simulate the joint positions, force applications and proprioceptive feedback of the natural foot in mid-stance gait and captures the topographical surface of the foot for orthosis production. The apparatus provides proper soft tissue compression while allowing an optical scanner to visualize the modified topographical skin surface. The apparatus includes an optically-transparent membrane to deform the soft tissue of the foot and an optical scanner configured to scan the foot through the membrane.

### BACKGROUND

a. Technical Field

This invention relates generally to an apparatus for capturing the shape of a foot for an orthosis and, more specifically, to an apparatus for optical scanning of a human foot for orthosis.

b. Background Art

There are several known types of methods for capturing the shape of the foot for orthosis. One type is a full weight bearing method where the surface of the foot is captured while the patient is standing. In embodiments, plaster cast material is wrapped around the foot, and often the patient stands on a foam block or a hard surface. In other embodiments, the patient stands on a clear glass surface and the weight-bearing image of the foot is scanned by an optical scanner through the glass. With full-weight-bearing methods, the soft tissue is excessively deformed so that there is little contour left, and the topographical surface becomes the same shape as the flat glass or weight bearing surface. An example of a soft tissue envelope 18 of a foot in a full-weight-bearing method is shown in FIG. 2A. Any resulting orthosis also has little contour, so considerable work must be done to modify the shape after capture to produce a foot orthosis that would be beneficial to the patient.

A second type of method is to capture the foot in a non-weight-bearing position. This may be accomplished using a suspension cast/capture technique where the foot is held in the correct segmental position to replicate mid-stance using one hand to position and hold the foot. This is often the ideal technique for positioning but is technically difficult and, being non-weight-bearing, provides little to no soft tissue deformation. An example of the soft tissue envelope 22 of a foot in a non-weight bearing position is shown in FIG. 2C. If a foot orthosis is fabricated from a non-weight-bearing surface without any modification, the resulting orthosis may be very uncomfortable due to normal physiological soft tissue deformation under body weight. Thus, some soft tissue deformation is beneficial while excessive or no deformation causes problems. To remedy this in a non-weight bearing technique, contours of the foot image or cast may be modified to allow for some soft tissue deformation or expansion in certain areas such as the fat pad of the heel, high point of the arch and muscle belly of the lateral intrinsic muscles.

A third type of method is a partial-weight-bearing capture. In this method only part of the body weight is utilized to compress the soft tissue while still allowing for proper foot segment positioning. An example of the soft tissue envelope 20 of a foot in a partial-weight-bearing technique is shown in FIG. 2B. An important aspect of partial-weight-bearing methods is proper soft tissue compression via the correct amount and distribution of load or weight on the leg and foot. Known partial-weight-bearing techniques utilize plaster cast material and foam blocks or, more recently, compressible foam that would crush under physiological loads. A drawback to foam impression systems is the lack of elasticity—i.e., once the foam is compressed, it generally cannot return to a non-compressed state, which does not allow any errors in positioning the foot.

### BRIEF SUMMARY

An embodiment of an apparatus for capturing the shape of a foot may include an optically-transparent flexible membrane configured to receive the foot, a support structure configured to retain the membrane, and an optical scanner configured to capture an image of the foot through the membrane. The membrane may comprise linear low-density polyethylene having a thickness of between about 60 mil and about 120 mil. The membrane may have a generally uniform thickness, or may have a variable thickness. In an embodiment, the optical scanner may be configured to capture an image of the foot from the side.

### BRIEF DESCRIPTION OF THE DRAWINGS

FIGS. 1A-1C are various views of the skeletal structure of a human foot.

FIGS. 2A-2C are diagrammatic views of the heel soft tissue envelope of a human foot under various weight-bearing conditions.

FIGS. 3A and 3B are side and bottom views of the skeletal structure of a human foot, illustrating functional axes of the foot.

FIG. 4 is a front view of the skeletal structure of a human foot, illustrating a first ray axis.

FIGS. 5A and 5B are cross-sectional views of a human foot in association with a membrane that may find use in an apparatus 40 capturing the shape of a foot.

FIG. 6 is an isometric view of an exemplary apparatus for capturing the shape of a foot.

FIGS. 7 and 8 are upper and lower isometric views, respectively, of the apparatus of FIG. 6 with a patient's foot disposed in a membrane of the apparatus.

FIG. 9 is an isometric view of an optical scanner of the apparatus of FIG. 6.

### DETAILED DESCRIPTION OF THE DRAWINGS

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation given that such combination is not illogical or non-functional.

Referring to the drawings, in which like numerals refer to the same or similar elements in the various drawings, FIGS. 1A-1C are various views of the skeletal structure of a human foot 10. There are several theories on how an orthosis for the foot 10 works, and therefore how to produce such an orthosis. One of the prominent theories is that the position of the joint segments in mid-stance of the gait cycle when the center of mass is over the foot 10. The position of the foot 10 is the key to allowing proper foot function prior to and following mid-stance.

There are 3 key functional joint segments that come into play in the foot 10 during ambulation. One is the ankle-subtalar joint complex 12. While the ankle and subtalar joint are separate anatomical joints, they work together in the positioning of the rear foot that comprises the talus and calcaneus bones. The second functional segment is the mid-tarsal joint 14, which determines the position if the forefoot. The mid-tarsal joint 14 also is two anatomical joints arising from the calcaneo-cuboid and talo-navicular articulations. The third functional segment is the first ray or medial column 16 of the foot that is comprised of the first metatarsal and medial cuneiform bones and their articulations. Together these functional segments 12, 14, 16 of the foot provide the complex three dimensional motions required for efficient ambulation.

To allow for the dynamic movement provided by the functional segments 12, 14, 16 of the foot 10 while using a static device such as a foot orthosis, one period of the gait cycle may be chosen as a reference point for the position and topographical surface shape of the orthosis, and the foot measured for an orthosis while in that position. One period that may be selected is mid-stance, when the ankle-subtalar joint 12 is in its “neutral” or central articulated position, the mid-tarsal joint 14 is in a “locked” or maximally pronated position and the medial column 16 of the foot 10 is plantarflexed. If the foot 10 is positioned in this mid-stance position and the topographical surface of the foot 10 is captured while in this position, that surface may be effectively replicated with an orthosis shell that, when placed under the foot, will press against the soft tissue that in turn pushes the osseous tissue to replicate the original position. Thus, it is important to capture the topographical shape of the foot with proper soft tissue deformation.

To overcome the deficiencies in known techniques for capturing a foot for producing an orthosis, a partially-elastic, clear membrane may be used to deform the soft tissue of the foot. The membrane may have some plastic memory (i.e., may be partially elastic), so as the joint is moved through its range of motion to find the proper neutral position, the membrane will not permanently be deformed. This property may offer an advantage over foam for a partial-weight-bearing procedure, as the foam may be inelastic (i.e., may collapse into a compressed state). In contrast, even when a partially-elastic membrane is stretched, the foot can still be manipulated while the soft tissue compression of the heel dynamically changes with the changing position of the proximal segment. This agrees with the theory of how a foot orthosis works. That is, the shape of the curvature of the heel may determine the reactive forces acting on the heel and proximal joint segments. Similarly, the theory is that as the proximal joints are positioned, the contour of the soft tissue will change. FIGS. 2A-2C illustrate the soft tissue envelope of the foot 10 under a full-weight-bearing procedure (i.e., shown as envelope 18), a partial-weight-bearing procedure (envelope 20), and a non-weight-bearing procedure (envelope 22). Once the joint segments are properly aligned, the topographical shape of the surface of the foot 10 may be captured. The topographic surface of the foot 10 may then be replicated with the orthotic material, thereby replicating the positional loads on the joints. A proper position for a foot for capturing its shape for an orthosis is further described below.

FIGS. 3A and 3B are side and bottom views of the skeletal structure of a human foot 10, illustrating functional axes of the foot 10. As the foot 10 starts to press down on a membrane such as that described above and shown and described with reference to FIGS. 6-9, the reactive compressive forces will dorsiflex the forefoot on the rearfoot. This dorsiflexion is desirable, as it replicates the foot in mid-stance of the gait cycle. When the forefoot dorsiflexes, the joint segment that is first to respond to allow the dorsiflexion is the mid-tarsal joint 14. The mid-tarsal joint 14 has two functional axes, the longitudinal axis 24 and oblique axis 26. The oblique axis 26 is more dominant in the sagittal plane than is the longitudinal axis 24, so as the foot dorsiflexes, tri-plane motion across the oblique axis 26 results, forcing the foot to pronate and, in this case, to its end range of motion (dorsiflexed-everted-abducted). This may be referred to as the “locked position,” as there is an osseous restraining mechanism in the mid-tarsal joint 14, creating inherent stability with increased joint congruity. The foot 10 may be placed in this “locked position,” in an embodiment, in a membrane for measuring the foot 10 for orthosis.

FIG. 4 is a front view of the skeletal structure of the foot 10, illustrating a first ray axis 28. In a partial-weight bearing procedure, the other segment of the foot 10 that may be manipulated is the first ray 30. Positioning of the first ray 30 may require manipulation by an expert, such as a physician, to determine the correct position. Generally, the first ray 30 should be positioned in a plantarflexed position as nearly as possible when capturing the shape of the foot 10 for an orthosis.

Similarly, the rotational axis of the metatarsal phalangeal joints 32 may be manipulated to prevent excessive dorsiflexion under the load of the membrane. A physician may push the metatarsal phalangeal joints 32 into the membrane (i.e., to deform the membrane and lessen the pressure on the joints) to avoid retrograde plantarflexion of the metatarsal heads.

FIGS. 5A and 5B are cross-sectional views of the foot 10 in association with a membrane 34 for an apparatus for capturing the shape of the foot 10. For the membrane 34 to provide proper soft tissue deformation of the foot 10, proper support of the membrane 34 may be important. Accordingly, a support structure for the membrane 34 may be designed or otherwise configured for a desired procedure, i.e., to produce a desired soft tissue deformation for a particular patient or type of patient. In an embodiment, the membrane 34 may be anchored superiorly around the foot 10 and the frame anchoring the membrane 34 may be a predetermined distance away from the foot 10 (i.e., away from the portion of the membrane in which the foot is intended to be pressed) to result in the correct angle of approach of the membrane 34 to the edges of the foot 10. In an embodiment, the support structure may couple with the membrane, perpendicular to the length of the foot, about 4.0-4.5 inches from the center of the membrane. FIGS. 5A and 5B illustrate minimal and maximal angles of approach, respectively, for an embodiment of a partial-weight bearing procedure involving a membrane 34. Too shallow of an angle may not provide proper soft tissue deformation, while too sharp of an angle may wrap the foot excessively with the membrane 30. Proper position of the membrane 30 may be, at a minimum, departing the surface of the foot at the plantar to dorsal skin demarcation. At a maximum, it may depart near the change in the radius of curvature between the dorsum of the foot to surrounding edges.

The support mechanism may be designed or otherwise configured to provide a selected uniform or variable tension on the foot 10 by the membrane 34. For example, the physical position of the support mechanism may be altered (e.g., shifted higher or lower) to increase or decrease the tension of the membrane against the foot. In addition, the shape of the support structure may be altered to provide more uniform or more variable tension. For example, in an embodiment, a generally rectangular support structure may be used to provide uniform tension. In other embodiments, a support structure having non-parallel sides may be used for variable tension, for example only. In any embodiment, secure edge connectors may be important—edge connectors slipping may unexpectedly reduce membrane tension.

In addition to the support structure, properties of the membrane 34 itself may be configured for a desired procedure. A membrane 34 having uniform thickness may be selected, in embodiments, to produce uniform tension and, thus, uniform pressure across the foot 10. In other embodiments, variable tensioning may be achieved by 1) varying the thickness of the membrane 34 material or 2) stretching the membrane 34 beyond the elastic region of the stress-strain curve of the membrane 34 and into the plastic region, thereby over-stretching and deforming the membrane 34 in areas to achieve proper tensioning of the membrane 34 against the foot 10. Variable tension in the membrane 34 may be desired to produce more compressive forces across some areas of the foot 10 while allowing for less compressive forces in other areas of the foot 10. In an embodiment, the area of the membrane 34 in which the patient's toes are to be depressed may have a decreased compressive load to minimize toe dorsiflexion while compressing the foot onto the membrane 34. In an embodiment, the medial column 16 or first ray 30 of the foot 10 may have reduced tension, allowing for plantarflexion, while the lateral column has greater upward loads to pronate or lock the mid-tarsal joint 14, while the medial rearfoot may have greater compressive force than the lateral rearfoot to invert (supinate) the rearfoot.

FIG. 6 is an isometric view of an exemplary apparatus 40 for capturing the shape of a foot. FIGS. 7 and 8 are upper and lower isometric views, respectively, of the apparatus 40 with a patient's foot 10 disposed in a membrane 34 of the apparatus 40. FIG. 9 is an isometric view of an optical scanner 42 of the apparatus 40. The apparatus 40 may be an optical scanning apparatus configured to provide proper compression of the soft tissue of the foot 10 to simulate joint positions, force applications, and proprioceptive feedback of the foot in mid-stance of a normal gait and may capture an image of the topographical surface of the foot 10 for orthosis. Referring to FIGS. 6-9, the apparatus 40 may include a support structure 44, the membrane 34, and an optical scanner 42 coupled with a track 46.

The support structure 44 may comprise, in an embodiment, an elliptical frame 48 and support arms 50 surrounding a rectangular portion 52. The rectangular portion 52 may include an upper surface 54 which may include edge connectors 56 configured to couple the membrane 34 with the support structure 44. The frame 48 may comprise a shape other than an oval, in an embodiment. Similarly, the rectangular portion 52 may comprise a shape other than a rectangle, in an embodiment. The support structure 44 may comprise materials and construction known in the art.

The membrane 34 may be optically transparent (e.g., generally transparent to the optical scanner 42), and may be made of a plastic or polymer film such as, for example only, linear low-density polyethylene (LLDPE) coupled with a membrane frame 60. The membrane frame 60 may include holes or other structures for coupling with the edge connectors 56. The membrane may be flexible and partially elastic. In an embodiment, the membrane may be 120 mil stretch wrap. In various embodiments, the membrane 34 may include thicknesses ranging from about sixty (60) mil to about one hundred-twenty (120) mil, for example only, though the membrane 34 is not limited. Thinner material (e.g., about 60 mil) may be preferred for lighter patients, and thicker material (e.g., about 120 mil) for heavier patients.

The membrane 34 may provide an advantage over known orthosis measurement devices. In known devices including optical scanners, a solid glass plate is often used which, as noted above, results in a full-weight-bearing procedure and improper soft tissue deformation. In other devices, if a membrane is included, the membrane generally is partially or completely opaque to the optical seamier. Accordingly, such devices generally measure the membrane itself, rather than the foot 10. Measuring the membrane, however, may make detection of the edge of the foot 10 difficult, which may create difficulties in properly sizing an orthosis based on the measurement. In contrast, an optically-transparent membrane 34 allows for the foot itself to be measured by the optical scanner 42.

The track 46 may be provided circumferentially about the frame 48 and below the upper surface 54 and below the membrane 34. The track 46 may be configured to guide a carrier 58 coupled with the optical scanner 42 to enable the optical scanner to image the entire foot 10 from the side of the foot 10. The movement of the optical scanner 42 about the foot 10 may allow a true three-dimensional image of the foot 10 to be captured.

In an exemplary procedure, a physician may attach a membrane 34 to the support structure 44 by placing the membrane frame 60 on the upper surface 54 of the rectangular portion 52 of the support structure 44. The physician may then press a patient's foot 10 down into the membrane 34 (e.g., the lateral center of the membrane 34) to place appropriate pressure on the patient's foot 10. The physician may flex the patient's foot 10 and the membrane 34 to place the foot 10 in the proper neutral position, substantially as described above. In an embodiment, the physician may press the foot 10 down such that the entire foot is below the upper surface 54. The physician may then activate the optical scanner 42. The carrier 58 (and, with it, the optical scanner 42) may orbit at least once about the entire circumference of the track 46, or a subset thereof as is necessary or directed. While conducting such an orbit or partial orbit, the optical scanner 42 may capture an image or a series of images of the foot 10, from the side of the foot 10, through the membrane 34. The optical scanner 42 may transmit a signal to a computer, which may compile the image or images captured of the foot 10 to create a three-dimensional rendering of the foot 10 as positioned. The three-dimensional rendering of the foot may be replicated, in an embodiment, to create an orthosis for the foot.

Although a number of embodiments have been described above with a certain degree of particularity, those skilled in the art could make numerous alterations to the disclosed embodiments without departing from the spirit or scope of this disclosure. For example, all joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of this disclosure as defined in the appended claims.

What is claimed is:

1. An apparatus for capturing the shape of a foot, said apparatus comprising:
an optically-transparent flexible membrane configured to receive the foot;
a support structure configured to retain said membrane; and
an optical scanner configured to capture an image of the foot through said membrane.

2. The apparatus of claim 1, further comprising a carrier coupled with said optical scanner and disposed on a track to convey said carrier.

3. The apparatus of claim 2, wherein said track is generally elliptical.

4. The apparatus of claim 3, wherein said track laterally surrounds said membrane.

5. The apparatus of claim 1, wherein said support structure comprises a rectangular portion configured to be coupled with said membrane.

6. The apparatus of claim 1, wherein said membrane comprises linear low-density polyethylene.

7. The apparatus of claim 6, wherein said membrane has a thickness of between about 60 mil and about 120 mil.

8. The apparatus of claim 7, wherein said membrane has a generally uniform thickness.

9. The apparatus of claim 7, wherein said membrane has a variable thickness.

10. The apparatus of claim 1, wherein said membrane has a generally uniform thickness.

11. The apparatus of claim 1, wherein said membrane has a variable thickness.

12. The apparatus of claim 1, wherein said membrane is generally rectangular.

13. The apparatus of claim 1, wherein said membrane comprises opposed sides that are not parallel.

14. An apparatus for capturing the shape of a foot, said apparatus comprising:
an optically-transparent flexible membrane configured to receive the foot;
a support structure configured to retain said membrane; and
an optical scanner configured to capture an image of the foot through said membrane, wherein said optical scanner is configured to capture the image from the side of the foot.

15. The apparatus of claim 14, wherein said support structure comprises an upper surface configured to secure said membrane to said support structure, further wherein said optical scanner is configured to capture an image or images of the foot below the upper surface.

16. The apparatus of claim 15, wherein said support structure comprises a rectangular portion comprising said upper surface, further wherein said membrane comprises a rectangular membrane support structure.

17. The apparatus of claim 14, wherein said support structure comprises edge connectors for securing said membrane to said support structure.

18. The apparatus of claim 14, wherein said membrane comprises linear low-density polyethylene.

19. The apparatus of claim 18, wherein said membrane has a generally uniform thickness.

20. The apparatus of claim 18, wherein said membrane has a variable thickness.
